SEQUENCE LISTING (2 sequences)

SEQ ID NO: 1 - DNA, 60 bp, artificial
  SEQ_0 203
gatttggggttcaaagcagtatcgatcaaatagtaaatccatttgttcaactcacagttt

SEQ ID NO: 2 - DNA, 60 bp, artificial; identical to SEQ ID NO: 1
  SEQ_1 203

(19) United States
(12) Patent Application Publication
(10) Pub. No.: US 2022/0172801 A1
(43) Pub. Date: Jun. 2, 2022

(54) Training Device, Disease Affection Determination Device, Classification Device, Machine Learning Method, and Classification Method

(71) Applicant: Preferred Networks, Inc., Tokyo (JP)

(72) Inventors: Nobuyuki Ota, Tokyo (JP); Shuji Suzuki, Tokyo (JP); Motoki Abe, Tokyo (JP)

(73) Assignee: Preferred Networks, Inc., Tokyo (JP)

(21) Appl. No.: 17/512,810

(22) Filed: Oct. 28, 2021

Related U.S. Application Data
(63) Continuation of application No. PCT/JP2020/003421, filed on Jan. 30, 2020.
(60) Provisional application No. 62/840,156, filed on Apr. 29, 2019.

(51) Int. Cl. G16B 40/20; G16B 20/20; G06N 20/00

(52) U.S. Cl. CPC G16B 40/20; G06N 20/00; G16B 20/20

(57) ABSTRACT

Provided are a training device, a disease affection determination device, a machine learning method, and a program that are applicable to various living organisms other than humans without performing time-consuming mapping. The present disclosure provides a machine learning unit that trains a model for a predetermined disease using, as an input, a training feature vector based on an appearance frequency of a plurality of types of substrings in a base sequence obtained from a training sample collected from a learning subject and, as an output, label information indicating whether the learning subject is a subject affected by the predetermined disease or a subject not affected by the predetermined disease.

### CROSS-REFERENCE TO RELATED APPLICATION

This is a continuation application of International Application No. PCT/JP2020/003421, with an International filing date of Jan. 30, 2020, which claims priority of U.S. Provisional Patent Application No. 62/840,156 filed on Apr. 29, 2019, the entire content of which is hereby incorporated by reference.

### TECHNICAL FIELD

The present disclosure relates to techniques of a training device, a disease affection determination device, a machine learning method, and a program.

### BACKGROUND ART

Conventionally, as a technique for determining cancer affection using RNA of blood or skin tissue, there has been developed a technique for measuring an expression level of a specific microRNA with a microarray or a DNA sequencer and determining cancer affection using the expression level of the microRNA as an input.

### CITATION LIST

### Non-Patent Literature

### Non-Patent Literature 1

Shimomura, A., Shiino, S., Kawauchi, J., Takizawa, S., Sakamoto, H., Matsuzaki, J., . . . Ochiya, T. (2016). Novel combination of serum microRNA for detecting breast cancer in the early stage. Cancer Science, 107(3), 326-34. https://doi.org/10. 1111/cas. 12880

### SUMMARY OF INVENTION

An aspect of a training device of the present disclosure includes a machine learning unit configured to train a model for a predetermined disease using, as an input, a training feature vector based on an appearance frequency of a plurality of types of substrings in a base sequence obtained from a training sample collected from a learning subject and, as an output, label information indicating whether the learning subject is a subject affected by the predetermined disease or a subject not affected by the predetermined disease.

### BRIEF DESCRIPTION OF DRAWINGS

FIG. 1 is a block diagram showing a schematic configuration of a disease affection determination device according to a first embodiment of the present disclosure.

FIG. 2 is a diagram showing a schematic hardware configuration of the disease affection determination device.

FIG. 3 is a flowchart showing a flow of processing in the disease affection determination device.

FIG. 4 is a diagram showing an example of RNA sequence data in a Fasta format.

FIG. 5 is a diagram showing an example of label information.

FIG. 6 is a view showing an example of creating k-mer.

FIG. 7 is a view showing an example of calculating an appearance frequency of the k-mer shown in FIG. 6.

FIG. 8 is a diagram illustrating an algorithm of a random forest.

FIG. 9 is a diagram showing an evaluation result in an example.

FIG. 10 is a diagram showing an example of creating a substring by spaced speed in a second embodiment of the present disclosure.

FIG. 11 is a diagram showing an example of creating a representative string using an error correcting code for a substring in the second embodiment of the present disclosure.

FIG. 12 is a diagram showing an example of label information in a third embodiment of the present disclosure.

FIG. 13 is a diagram showing another example of label information in the third embodiment of the present disclosure.

FIG. 14 is a block diagram showing an example of a hardware configuration in an embodiment of the present disclosure.

FIG. 15 is a block diagram showing a schematic configuration of another disease affection determination device according to the first embodiment of the present disclosure.

FIG. 16 is a flowchart showing a flow of processing in another disease affection determination device shown in FIG. 15.

### DESCRIPTION OF EMBODIMENTS

According to an aspect of the present disclosure, a machine learning unit obtains an appearance frequency of a plurality of types of substrings in a base sequence obtained from a training sample collected from a learning subject. In addition, the machine learning unit uses a training feature vector based on the appearance frequency. Further, the machine learning unit trains a model using the training feature vector as an input and label information as an output indicating whether the learning subject is a subject affected by a predetermined disease or a subject not affected by the predetermined disease. Accordingly, it is possible to obtain a model for determining disease affection for a predetermined disease due to a genetic mutation without time-consuming mapping. In addition, since mapping is not performed, it is possible to obtain a model for determining disease affection for a predetermined disease due to a genetic mutation, for various living organisms other than humans.

Embodiments of a disease affection determination device according to the present disclosure will be described with reference to the accompanying drawings.

### First Embodiment

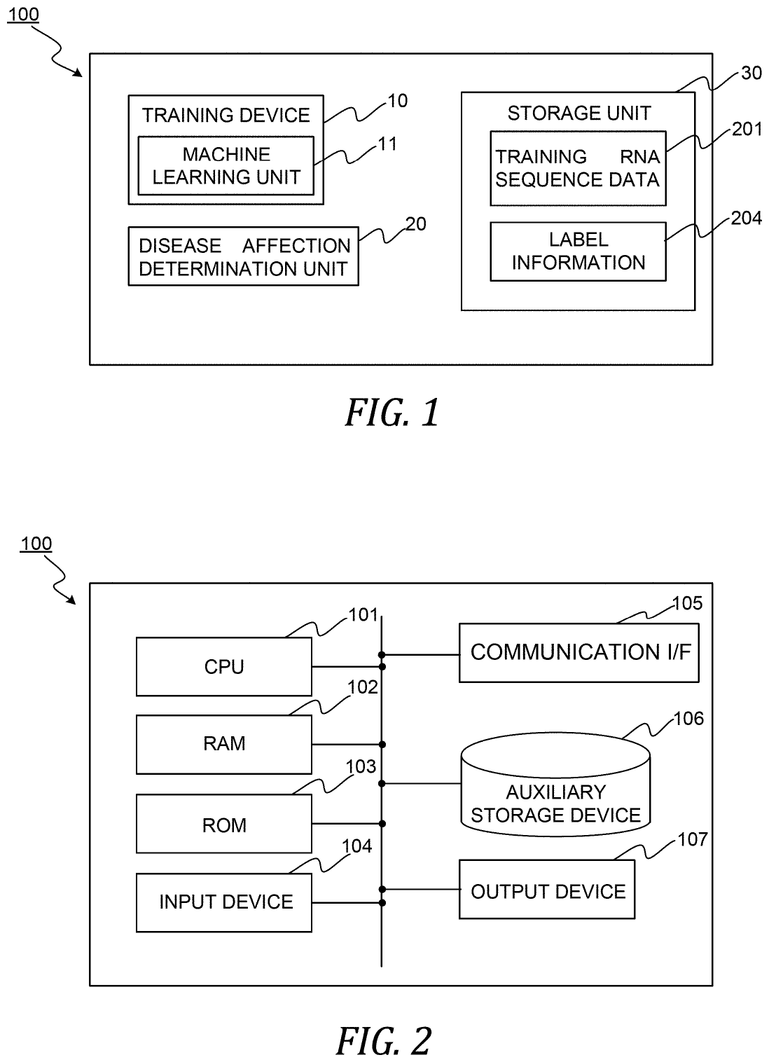

First, a first embodiment of a disease affection determination device according to the present disclosure will be described with reference to FIGS. 1 to 9.

### <Schematic Configuration of Disease Affection Determination Device>

FIG. 1 is a block diagram showing a schematic configuration of a disease affection determination device according to a first embodiment. As shown in FIG. 1, a disease affection determination device 100 of the present embodiment includes a training device 10 as a classification device, a disease affection determination unit 20, and a storage unit 30.

The training device 10 of the present embodiment includes a machine learning unit 11. The machine learning unit 11 obtains a training feature vector for a predetermined disease (clinical condition). In the present embodiment, cancer is chosen as a predetermined disease, and examples of learning subjects include a subject affected by the cancer and a subject not affected by the cancer. The learning subjects (reference subjects) may be humans or animals other than the humans. The machine learning unit 11 obtains an appearance frequency of a plurality of types of substrings in a base sequence obtained from training samples collected from such a learning subject. Then, a training feature vector is obtained based on the obtained appearance frequency. Further, the machine learning unit 11 trains a model using the training feature vector as an input and label information indicating whether the learning subject is in a clinical condition of affected by a predetermined disease or in a clinical condition of not affected by the predetermined disease, as an output.

The disease affection determination unit 20 of the present embodiment uses, as an input, a feature vector for determination based on an appearance frequency of a substring in a base sequence obtained from a biological sample for determination collected from a determination subject to perform a disease affection determination on the determination subject. In other words, the disease affection determination unit 20 outputs whether the determination subject is affected by a predetermined disease, using the appearance frequency of the substring in the base sequence obtained from the determination subject, as an input. Similarly to the learning subject, the determination subject may be a human or an animal other than the human.

The storage unit 30 of the present embodiment stores training RNA sequence data 201 to be described below and label information 204 to be described below. In addition, the storage unit 30 may store the model trained by the machine learning unit 11.

FIG. 2 is a diagram showing a schematic hardware configuration of the disease affection determination device 100 of the present embodiment. The disease affection determination device 100 has the same hardware as that of a normal information processing unit in terms of a basic configuration. For example, as shown in FIG. 2, the disease affection determination device 100 includes a CPU 101, a RAM 102, a ROM 103, and an input device 104 such as a keyboard or a mouse. Further, the disease affection determination device 100 includes a communication interface 105 that communicates with the outside, an auxiliary storage device 106 such as a hard disk, and an output device 107 such as a display or a printer.

### <Processing in Disease Affection Determination Device>

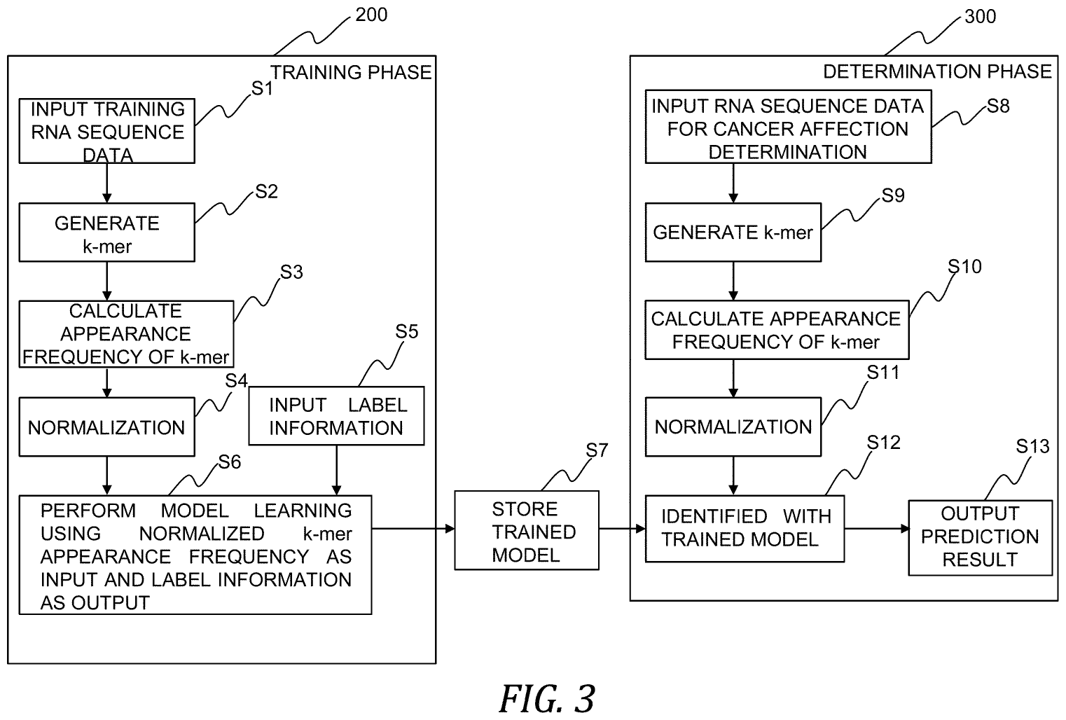

Next, a flow of processing in the disease affection determination device 100 will be described with reference to FIG. 3. FIG. 3 is a flowchart showing the flow of processing in the disease affection determination device 100 of the present embodiment.

As shown in FIG. 3, the processing in the disease affection determination device 100 of the present embodiment is divided into, for example, a training phase 200 and a determination phase 300. First, the training phase 200 will be described.

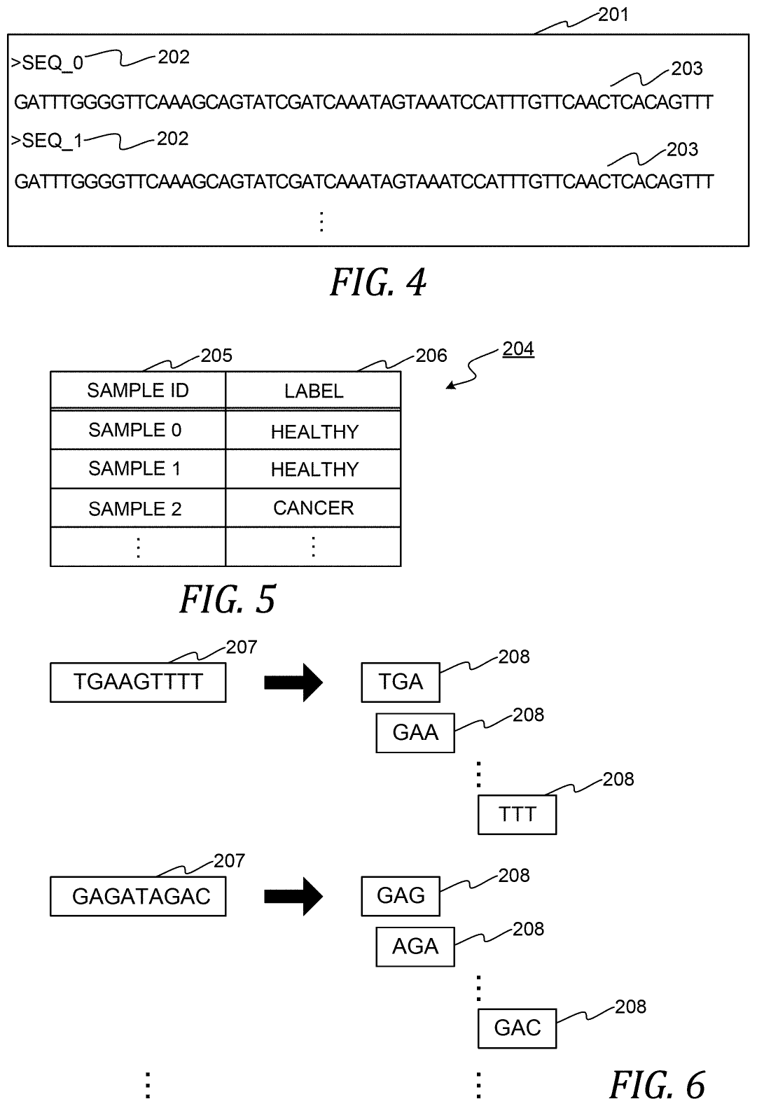

In the present embodiment, the RNA sequence data 201 is used as training data. The RNA sequence data 201 is stored in the storage unit 30 as an example. The RNA sequence data 201 is acquired, as a DNA sequence, from RNA of a biological sample (blood, saliva, or sebum) collected from a subject affected by cancer and a healthy subject, using a DNA sequencer. As a data format of the RNA sequence data 201, for example, both of a Fasta format and a Fastq format can be used. As an example, an example of RNA sequence data 201 in a standard Fasta format is shown in FIG. 4. FIG. 4 is a diagram showing an example of the RNA sequence data 201 in the Fasta format.

The Fasta format is plain text. One RNA sequence data is composed of a header line 202 in a line, which begins with a symbol “>”, and actual sequence strings 203 in a second line and subsequent lines. In the header line 202, an ID is described next to the symbol “>” to identify sequence data. In FIG. 4, as an example, IDs of SEQ_0 and SEQ_1 are described.

Followed by the string for identifying the sequence data, a string (a sequence read which will be hereinafter referred to as a read) representing a base sequence read by a DNA sequencer is described as the sequence string 203. In FIG. 4, as an example, a read beginning with GATTT . . . is described.

When another line beginning with a symbol “>” appears followed by the sequence string 203, the sequence data is delimited and another sequence data begins.

In the present embodiment, as label information of the RNA sequence data 201, label information 204 is used as shown in FIG. 5. FIG. 5 is a diagram showing an example of label information in the present embodiment. As shown in FIG. 5, the label information 204 is a file in which a sample ID 205 assigned to each biological sample is paired with a label 206 indicating whether a biological sample identified by the sample ID 205 is a subject affected by cancer or a healthy subject. In FIG. 5, the sample IDs 205 of “Sample 0” and “Sample 1” are paired with the labels 206 “Healthy”, respectively, indicating that theses biological samples are healthy subjects. In addition, the sample ID 205 of “Sample 2” is paired with the label 206 “Cancer”, indicating that the biological sample is a subject affected by cancer. The label information 204 is stored in the storage unit 30 as an example.

In the training phase 200 of the present embodiment, the RNA sequence data 201 as described above and the label information 204 corresponding to the RNA sequence data 201 are used. In the present embodiment, the machine learning unit 11 converts the RNA sequence data 201 into a training feature vector by the following procedure.

(1) First, the machine learning unit 11 inputs the training RNA sequence data 201 (S1 in FIG. 3). The machine learning unit 11 may input the training RNA sequence data 201 stored in storage unit 30 in advance from the storage unit 30, or may input training RNA sequence data 201 from an external storage medium.

After inputting the training RNA sequence data 201, the machine learning unit 11 may perform error checking and post-processing of the DNA sequencer, or may perform predetermined processing of deleting a part having many errors in the RNA sequence data itself from the RNA sequence data 201. For example, the machine learning unit 11 may perform trimming based on a quality score, which is reading reliability of DNA output from the DNA sequencer, or may remove the RNA sequence data 201 in which the sequence is exactly the same. Further, when reading RNA with a DNA sequencer, the machine learning unit 11 may remove an adapter sequence assigned to the RNA.

(2) Next, the machine learning unit 11 generates k-mer for each read from the RNA sequence data 201 input in the Fasta format (S2 in FIG. 3). The k-mer is a substring composed of a continuous base (nucleic acid residue) obtained by cutting out the read output by the DNA sequencer for each number of characters k (k being an integer of 1 or more). The number of characters k can be set to any number. In the present embodiment, an example of k=3 will be described.

FIG. 6 shows an example of creating the k-mer. FIG. 6 is a view showing an example of creating the k-mer in the present embodiment. In the example shown in FIG. 6, k-mers 208 called “TGA”, “GAA” . . . , and “TTT” are created from a read 207 called “TGAAGTTTT”. Further, k-mers called “GAG”, “AGA” . . . , and “GAC” are created from a read 207 called “GAGATAGAC”.

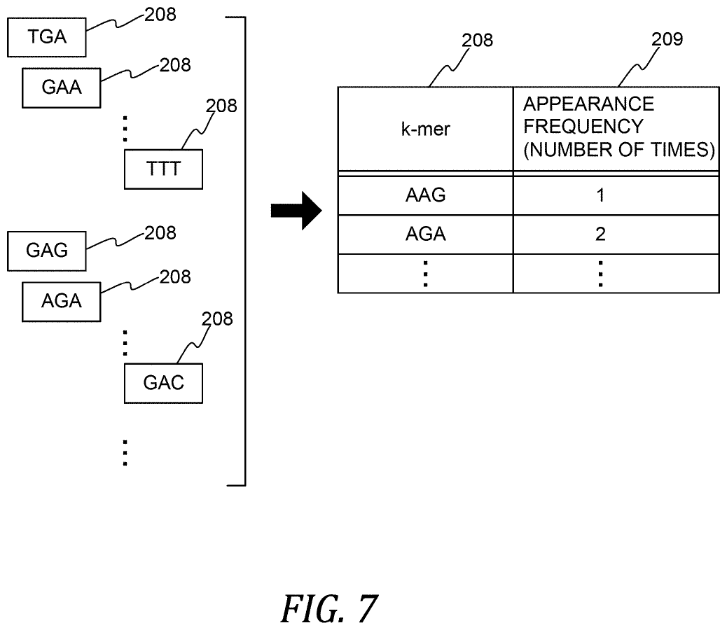

(3) Next, the machine learning unit 11 calculates an appearance frequency (number of times) of each k-mer per sample (S3 in FIG. 3). FIG. 7 is a view showing an example of calculating an appearance frequency of the k-mers shown in FIG. 6. In the example shown in FIG. 7, an appearance frequency 209 of a k-mer 208 called “AAG” is calculated to be once, and an appearance frequency 209 of a k-mer 208 called “AGA” is calculated to be twice.

(4) Next, the machine learning unit 11 normalizes the appearance frequency 209 of the k-mer 208 for each sample using following Formulas (S4 in FIG. 3). Even in the RNA sequence data 201 of the same sample, the number of reads 207 may be different, and as a result, the appearance frequency 209 of the k-mer 208 may be changed. Therefore, by the normalization, the difference in the appearance frequency 209 of the k-mer 208 due to the difference in the number of reads 27 can be corrected, and the appearance frequency can be appropriately determined.

$$\hat{{c}_{i , j}} = \frac{{c}_{i , j}}{{Σ}_{j} {c}_{i , j}} \qquad [ Formula 1 ]$$

In the above formula, C_{î,j} represents a normalized appearance frequency of a j-th k-mer in a sample i.

c_{i,j} represents an appearance frequency of the j-th k-mer in the sample i.

A denominator on the right side of the above formula represents an appearance frequency of all k-mers in the sample i.

(5) Next, the machine learning unit 11 inputs the label information 204 stored in advance in the storage unit 30 (S5 in FIG. 3). The machine learning unit 11 may input the label information 204 from an external storage medium or the like.

(6) Next, the machine learning unit 11 trains a model using the appearance frequency 209 of the normalized k-mer 208 in all the samples as described above and the label information 204 corresponding to all the samples (S6 in FIG. 3). At this time, as the model, a linear classification, a decision tree, an SVM, a random forest, and a multilayer perceptron can be used.

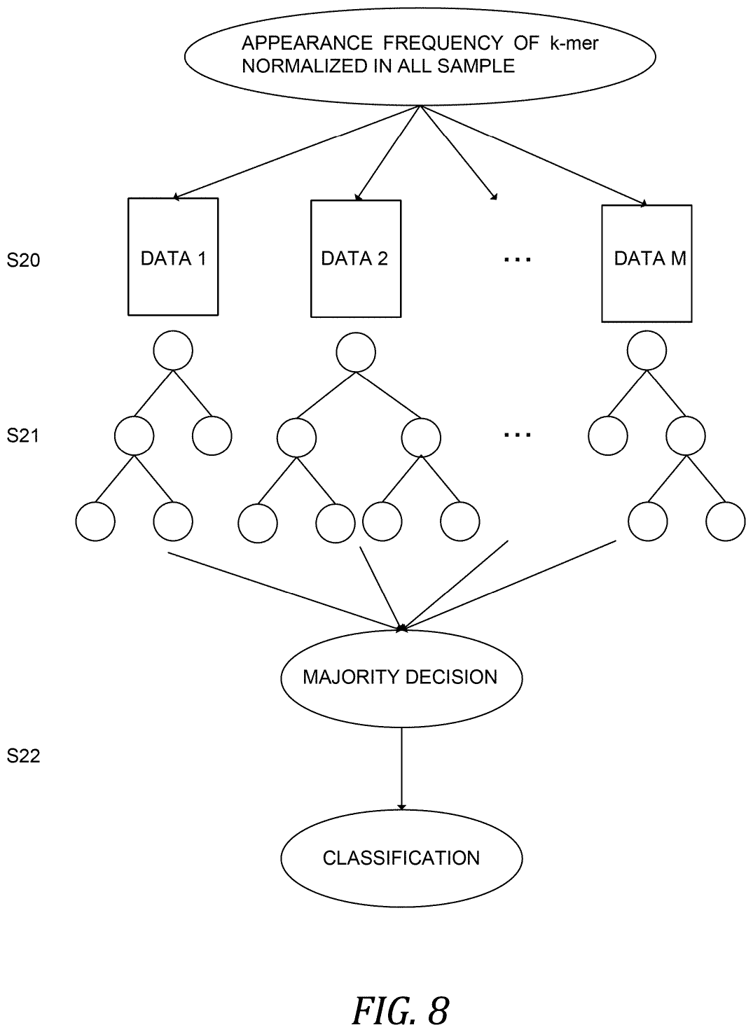

FIG. 8 is a diagram illustrating an algorithm of a random forest. As shown in FIG. 8, the appearance frequency 209 of the normalized k-mer 208 in all the samples is used as training data, and M (M being an integer of 1 or more) bootstrap specimens are extracted from, for example, two thirds of the total training data in step S20. M indicates a size of a forest. A size n (n being an integer of 1 or more) of one bootstrap specimen is, in principle, a size of the training data (⅔ of the total), for example. The remaining ⅓ is left as evaluation/verification data.

In step S21 shown in FIG. 8, after the appearance frequency 209 of all the k-mers 208 is selected as all variables in each bootstrap specimen and the appearance frequency 209 of d (d being an integer of 1 or more) k-mers 208 is selected, as d explanatory variables, in a random manner from the all variables, a subject affected by cancer and a healthy subject are classified, and a decision tree is grown. The number of explanatory variables can be set as appropriate.

In step S22 shown in FIG. 8, results of the obtained decision trees are integrated. In the present embodiment, the results are integrated by majority decision, a subject affected by cancer and a healthy subject are classified, and a training machine is constructed as a trained classification. The model constructed from the training data is applied to the evaluation/verification data to obtain an estimation error. In the present embodiment, for example, an erroneous discrimination rate is used as an index. From the estimation error, a correlation between the appearance frequency 209 of the k-mer 208 as an explanatory variable and the subject affected by cancer and the healthy subject can be obtained.

(7) The machine learning unit 11 stores the model trained as described above in the storage unit 30 as a trained model (S7 in FIG. 3).

Next, the determination phase 300 of the present embodiment will be described. In the present embodiment, the disease affection determination unit 20 converts the RNA sequence data 201 for performing the cancer affection determination into a feature vector for determination according to the following procedure, and the disease affection determination unit 20 performs disease affection determination as follows.

(1) First, the disease affection determination unit 20 inputs RNA sequence data (hereinafter, referred to as RNA sequence data for affection determination) for performing cancer affection determination (S8 in FIG. 3). The disease affection determination unit 20 may input the RNA sequence data for disease affection determination previously stored in the storage unit 30 from the storage unit 30, or may input the RNA sequence data for disease affection determination from an external storage medium.

(2) Next, the disease affection determination unit 20 generates k-mer 208 for each read from the RNA sequence data for disease affection determination input in the Fasta format (S9 in FIG. 3). In the present embodiment, an example of k=3 will be described as in the training phase.

(3) Next, the disease affection determination unit 20 calculates an appearance frequency (number of times) of each k-mer 208 per sample for disease affection determination (S10 in FIG. 3).

(4) Next, the disease affection determination unit 20 normalizes the appearance frequency 209 of the k-mer 208 for each sample for disease affection determination, using the above formula used in the training phase (S11 in FIG. 3). The reason for normalization is the same as described in the training phase.

(5) Next, the disease affection determination unit 20 inputs the appearance frequency 209 of the k-mer 208 normalized as described above in the sample for disease affection determination, and identifies the input appearance frequency from the trained model stored in the storage unit 30 (S12 in FIG. 3). Then, the disease affection determination unit 20 predicts whether the sample for disease affection determination is a subject affected by cancer or a healthy subject, and outputs prediction results (S13 in FIG. 3).

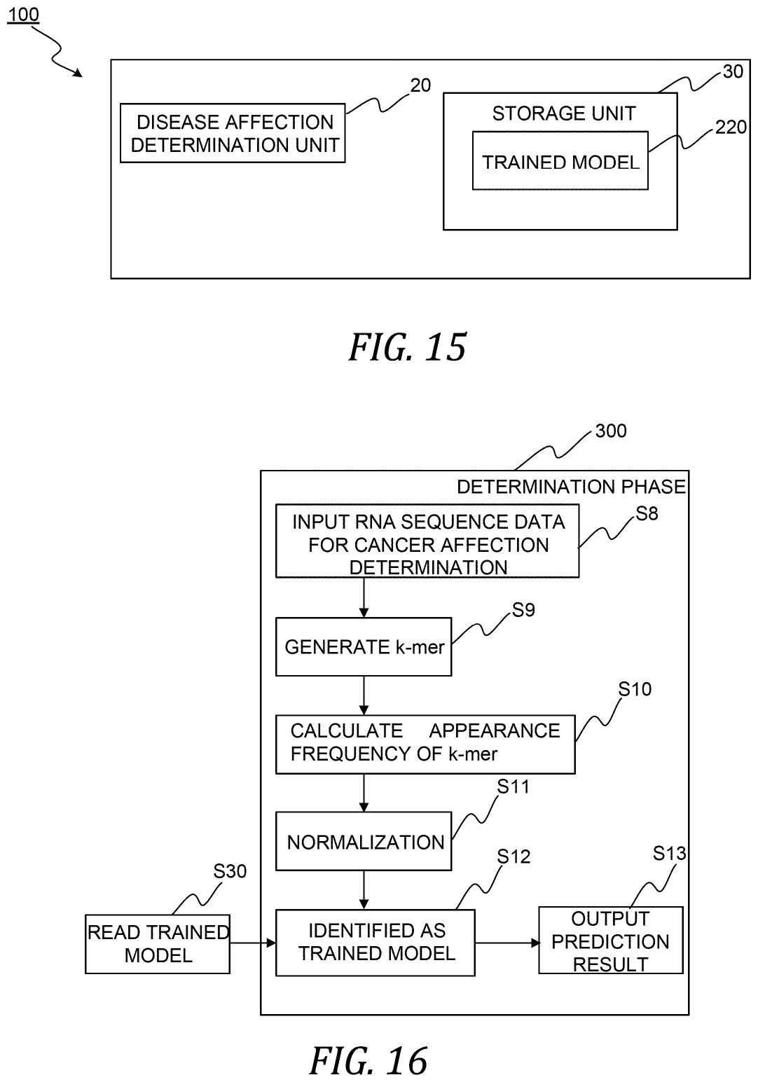

In the disease affection determination device 100 of the present disclosure, as shown in FIG. 15, an already trained model 220 is stored in the storage unit 30, and the trained model 220 can be used. In other words, the disease affection determination device 100 may include a disease affection determination unit 20 that can use the trained model 220, and perform a determination phase. In this case, it is not necessary to provide the machine learning unit 11, and it is not necessary to perform the training phase. As shown in the flowchart of FIG. 16, the disease affection determination unit 20 reads the trained model 220 from the storage unit 30 (S30 in FIG. 16), and executes the determination phase 300 (S8 to S13 in FIG. 16)

### EXAMPLE

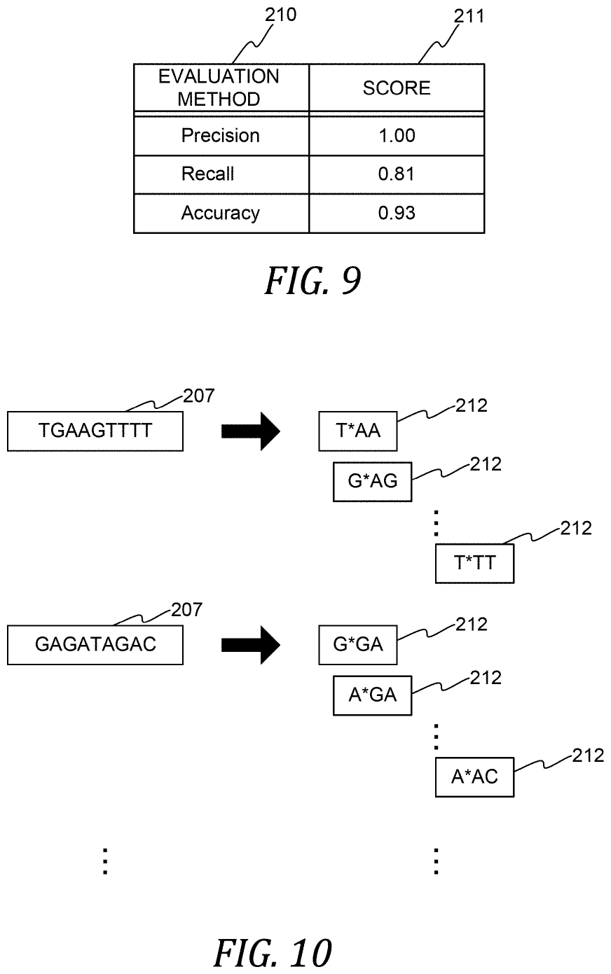

Next, a description will be given with respect to an example performed for verifying the disease affection determination device 100 of the present embodiment. In the example, 96 blood samples of healthy dogs and 52 blood samples of dogs affected by cancer were prepared, respectively, and read with a DNA sequencer. Then, the read samples were divided into 118 samples and 30 samples for training and verification, the training was performed using 118 samples, and evaluation was performed on the remaining 30 samples. As a training model, a random forest was used. The result of the evaluation is shown in FIG. 9. FIG. 9 is a diagram showing the evaluation result in the example.

As shown in FIG. 9, as evaluation methods 210, three methods of Precision, Recall, and Accuracy were used. These evaluation methods are obtained by the following patterns.

It is defined as a True Positive (TP) if the disease affection determination device 100 determines that the sample is a subject affected by cancer, and the sample is the subject actually affected by cancer. It is defined as a False Positive (FP) if the disease affection determination device 100 determines that the sample is the subject affected by cancer, but the sample is a subject is actually healthy subject. In addition, it is defined as a False Negative (FN) if the disease affection determination device 100 determines that the sample is the healthy subject, but the sample is a subject actually affected by cancer. It is defined as a True Negative (TN) if the disease affection determination device 100 determines that the sample is the healthy subject, and the sample is actually the healthy subject

When the evaluation patterns are defined as described above, a score 211 of each of the evaluation methods 210 is obtained as follows.

$$Precision = TP \text{/} ( TP + FP )$$

$$Recall = TP \text{/} ( TP + FN )$$

$$Accuracy = ( TP + TN ) \text{/} ( TP + TN + FP + FN )$$

As shown in FIG. 9, the score 211 was 1.00 when the evaluation method 210 is the Precision, the score 211 was 0.81 when the evaluation method 210 is the Recall, and the score 211 was 0.93 when the evaluation method 210 is the Accuracy.

As described above, according to the disease affection determination device 100 of the present embodiment, it can be seen that when the evaluation method 210 is the Accuracy, the cancer affection can be determined with high accuracy.

As described above, according to the present embodiment, the appearance frequency of k-mer as the plurality of types of substrings is obtained in the RNA sequence data which is the base sequence of the training sample, and the training feature vector based on the appearance frequency of the k-mer is used. Further, the appearance frequency of k-mer as the plurality of types of substrings is obtained in the RNA sequence data which is the base sequence obtained from the determination sample, and the feature vector for determination based on the appearance frequency of the k-mer is used. In the present embodiment, the feature vector for determination is used as an input to determine the disease affection of the determination subject.

Accordingly, in the present embodiment, the RNA sequence data is used for the cancer affection determination, but the RNA is not required to be mapped, that is, it is not necessary to calculate which gene or which microRNA is expressed how much, and calculation time can be shortened.

In addition, conventionally, since mapping is not originally possible in a case of organisms other than humans having no reference genome, there is a problem that the expression level of microRNA cannot be measured. However, according to the present embodiment, since mapping of RNA is not required, a reference genome is not necessary, and it can be applied to various organisms other than humans.

### Second Embodiment

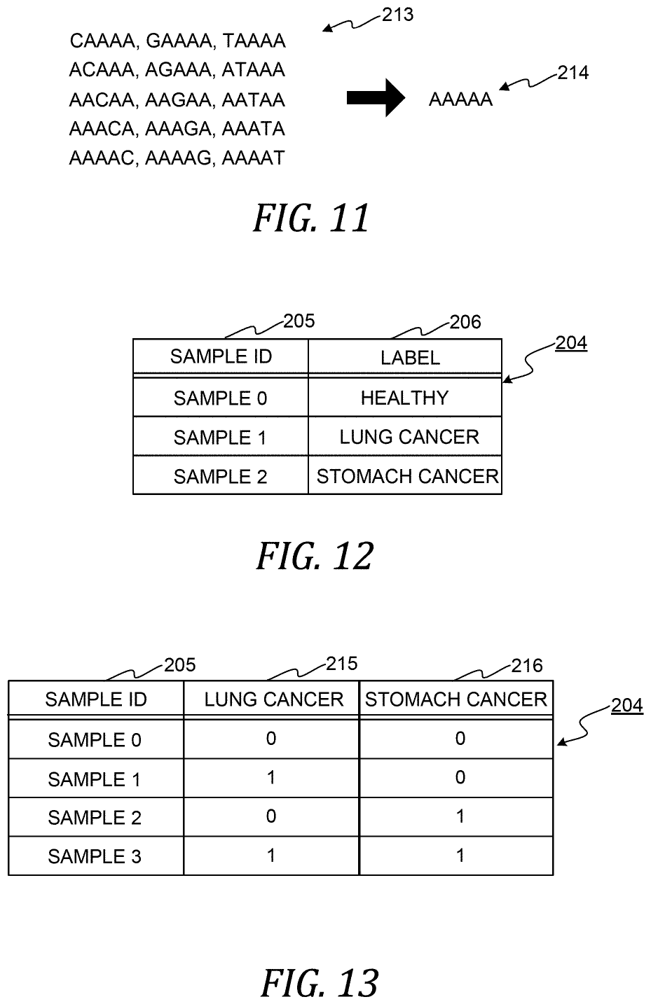

Next, a second embodiment of the present disclosure will be described with reference to FIGS. 10 and 11. FIG. 10 is a diagram showing an example of creating a substring by spaced speed in the present embodiment. FIG. 11 is a diagram showing an example in which a 4-ary (5,3) Hamming code being one of error correcting codes is applied to a substring created by a k-mer or a spaced seed of length 5.

The generation of k-mer described in the first embodiment corresponds to a case of calculating a substring from the string of the input RNA sequence data. There are various methods to generate such a substring, and the following methods can be used instead of k-mer:

(1) A method (spaced seed) of generating a string by skipping some characters (partial characters), instead of a continuous string;

In the k-mer, a continuous k-character substring was used. On the other hand, in the spaced seed, a spaced seed pattern formed by symbols 1 and 0 is defined in advance, and new strings are generated in order using only characters of the symbol 1. The k-mer corresponds to a case where all the spaced seed patterns are formed by the symbol 1.

FIG. 10 shows an example of creating a string in a case where the spaced seed pattern is “1011”. When the spaced seed pattern is “1011”, since a second character is 0, the second character is skipped. In FIG. 10, a part of “*” in a substring 212 created from a read 207 represents the skipped character. In the example shown in FIG. 10, substrings 212 called “T*AA”, “G*AG” . . . , and “T*TT” are created from a read 207 called “TGAAGTTTT”. Further, substrings 212 called “G*GA”, “A*GA” . . . , and “A*AC” are created from a read 207 called “GAGATAGAC”.

By skipping some characters in this way, it is possible to match a part of the strings generated from similar sequences. Thus, it is possible to make the disease affection determination robust against difference in RNA sequences due to individual differences in samples and sequencing errors.

(2) A method of converting a partially different string to the same string using an error correcting code for the substring created by the k-mer or the spaced seed.

Even when the spaced seed is used, it is possible to cope with difference in RNA sequences due to individual differences in samples and sequencing errors to some extent, but when the error correcting code is applied in addition to the spaced seed, differences in some characters, for example, several characters can be further absorbed.

The error correcting code is a technique for correcting an erroneous part of a sequence containing an error and converting it into a correct sequence. By an application of such a technique, a string with differences in some characters, for example, several characters can be converted into any representative string.

FIG. 11 is a diagram showing an example in which a 4-ary (5,3) Hamming code being one of error correcting codes is applied to a substring created by a k-mer or a spaced seed of length 5. As shown in FIG. 11, for example, when a substring 213 created by the k-mer or the spaced seed of length 5 is generated, an example is described in which the 4-ary (5,3) Hamming code being one of error correcting codes is applied to the substring 213. In this case, the substring 213 created by the k-mer or the spaced seed contains substrings such as CAAAA and AATAA, but such a substring is converted into AAAAA as a representative string 214 by an application of the 4-ary (5,3) Hamming code.

By such a process, it is possible to make the disease affection determination more robust against difference in RNA sequences due to individual differences in samples and sequencing errors compared with the case of the application of the spaced seed.

### Third Embodiment

Next, a third embodiment of the present disclosure will be described with reference to FIGS. 12 and 13. FIG. 12 is a diagram showing an example of label information in the present embodiment, and FIG. 13 is a diagram showing another example of label information in the present embodiment.

In the first embodiment, a binary classification of healthy or cancer has been performed. However, when cancer occurs, you may want to know where the cancer occurs. In order to cope with this, the present embodiment is configured such that when cancer occurs, any site where the cancer occurs can be predicted. In other words, the input is classified into a plurality of types.

FIG. 12 shows an example of label information 204 in which each sample ID 205 in the present embodiment is paired with a label indicating a site where cancer occurs. As shown in FIG. 12, the label information 204 is a file in which a sample ID 205 assigned to each biological sample is paired with a label 206 indicating whether the biological sample identified by the sample ID 205 is a healthy subject or, when the subject is affected by cancer, indicating a site where the cancer occurs. In FIG. 12, the sample ID 205 of “Sample 0” is paired with a label 206 called “Healthy”, indicating that the biological sample is healthy subject. In addition, the sample ID 205 of “Sample 1” is paired with a label 206 called “Lung cancer”, indicating that the biological sample is a subject affected by cancer and the cancer occurs in the lung. Further, the sample ID 205 of “Sample 2” is paired with a label 206 called “Stomach cancer, indicating that the biological sample is a subject affected by cancer and the cancer occurs in the stomach.

In this case, by using multi-class learning at the time of training the model, it is possible to collectively predict whether the sample is a healthy subject, or when the sample is a subject affected by cancer, to predict any site where the cancer occurs. In addition, types of tumors are divided into benign tumors and malignant tumors (cancers) and assigned with the label 206, and thus a model can be used in which the benign and malignant tumors are also determined discriminatingly.

In the above example, it is assumed that each sample is affected by only one type of cancer. However, the subject may be affected by a plurality of types of cancer due to metastatic cancer. In this case, the disease affection determination can be performed using the same method as described above by the change of the method of creating the label of the sample data.

FIG. 13 shows an example of label information corresponding to a case where a subject is affected by lung cancer and stomach cancer. In the example shown in FIG. 13, a label 215 corresponding to lung cancer and a label 216 corresponding to stomach cancer are used. The label 215 is set to 1 when the subject is affected by the lung cancer, and the label 215 is set to 0 when the subject is not affected by the lung cancer. Further, the label 216 is set to 1 when the subject is affected by the stomach cancer, and the label 216 is set to 0 when subject is not affected by the stomach cancer.

Therefore, when the subject is affected by both of the lung cancer and the stomach cancer, both the label 215 corresponding to the lung cancer and the label 216 corresponding to the stomach cancer are set to 1. In addition, when the subject is affected by either the lung cancer or the stomach cancer, either the label 215 corresponding to the lung cancer or the label 216 corresponding to the stomach cancer is set to 1. Further, when the subject is healthy, both the label 215 corresponding to the lung cancer and the label 216 corresponding to the stomach cancer are set to 0.

In this case, by using multi-label learning at the time of training the model, either the sample of the healthy subject or the sample of the subject affected by both lung cancer and stomach cancer or the subject affected by either lung cancer or stomach cancer when the sample is the subject affected by cancer can be collectively used as a predictable model.

In the example shown in FIG. 13, the sample ID 205 of “Sample 0” is paired with the label 215 of the lung cancer and the label 216 of the stomach cancer which are set to 0, indicating that the biological sample is a healthy subject. The sample ID 205 of “Sample 1” is paired with the label 215 of the lung cancer set to 1 and the label 216 of the stomach cancer set to 0, indicating that the biological sample is a subject affected by one type of cancer called lung cancer. The sample ID 205 of “Sample 2” is paired with the label 215 of the lung cancer set to 0 and the label 216 of the stomach cancer set to 1, indicating that the biological sample is a subject affected by one type of cancer called stomach cancer. The sample ID 205 of “Sample 3” is paired with the label 215 of the lung cancer and the label 216 of the stomach cancer which are both set to 1, indicating that the biological sample is a subject affected by two type of cancers called lung cancer and stomach cancer.

This method is a method called a multi-label. According to this method, the training sample data is applied with the labels indicating a plurality of types of different cancer affection, and the machine learning as described above is performed to create a trained model, whereby the disease affection determination can be performed for one or more cancers by one determination. In addition, as in the multi-class learning, types of tumors are divided into benign tumors and malignant tumors (cancers) and assigned with labels, and thus the benign and malignant tumors can also be determined discriminatingly.

### (Modifications)

In the embodiments described above, the cancer from a primary site is taken as an example of a clinical condition, and aspects have been described in which the present disclosure is applied to the cancer affection determination. However, the present disclosure is also applicable to cancers from two or more common primary sites, for example. An example of the cancer, to which the present disclosure is applicable, include breast cancer, lung cancer, prostate cancer, colorectal cancer, renal cancer, uterine cancer, pancreatic cancer, esophageal cancer, lymphoma, head/neck cancer, ovarian cancer, hepatobiliary cancer, melanoma, cervical cancer, multiple myeloma, leukemia, thyroid cancer, bladder cancer, stomach cancer, or a combination thereof.

In addition, the clinical condition in the present disclosure may be a predetermined stage of breast cancer, a predetermined stage of lung cancer, a predetermined stage of prostate cancer, a predetermined stage of colorectal cancer, a predetermined stage of renal cancer, a predetermined stage of uterine cancer, a predetermined stage of pancreatic cancer, a predetermined stage of esophageal cancer, a predetermined stage of lymphoma, a predetermined stage of head/neck cancer, a predetermined stage of ovarian cancer, a predetermined stage of hepatobiliary cancer, a predetermined stage of melanoma, a predetermined stage of cervical cancer, a predetermined stage of multiple myeloma, a predetermined stage of leukemia, a predetermined stage of thyroid cancer, a predetermined stage of bladder cancer, or a predetermined stage of stomach cancer.

In addition, the clinical condition in the present disclosure may be a predetermined subtype of cancer. Further, the present disclosure is also applicable to disease affection determination for another disease, for example, a disease caused by an abnormality of a hormonal system as a clinical condition. In particular, the present disclosure is appropriately applicable to disease affection determination for a disease caused by a mutation in a DNA sequence such as a genetic mutation. Here, the mutation in the DNA sequence such as the genetic mutation means that the expression level of microRNA is different from that of the healthy subject. In addition, the present disclosure is also applicable to a case of detecting DNA of microorganisms and determining an infectious disease.

The clinical condition in the present disclosure also includes a healthy state.

As the biological sample in the present disclosure, blood, whole blood, lymph, serum, saliva, urine, cerebrospinal fluid, fine needle aspiration fluid, tissue specimen, breast milk, nipple discharge, or in vitro fluid of a determination subject may be used.

In the first embodiment described above, an aspect has been described in which the disease affection determination is performed by the trained model after the model is trained. However, the present disclosure may be, for example, a disease affection determination device that determines disease affection using the trained model prepared by pre-training.

In the embodiments described above, the plurality of sequence reads can be obtained from single-ended next-generation sequencing or pair-ended next-generation sequencing for the biological sample of the determination subject.

In the embodiments described above, a case of k=3 has been described as an example of k-mer, but any one of 1, 2, 3, 4, 5, 6, 7, 8, 9, 10, 11, 12, and 15 can be used as a value of k.

In the embodiments described above, an example has been described in which 118 samples are used as a learning subject (reference subject), but at least 20 samples or at least 100 samples are applicable.

As the trained model as a trained classification, a neural network algorithm, a support vector machine algorithm, a decision tree algorithm, an unsupervised clustering model algorithm, a supervised clustering model algorithm, or a regression model can be used.

In the disease affection determination device 100 in the embodiments described above, each function may be implemented by a circuit constituted by an analog circuit, a digital circuit, or an analog/digital mixed circuit. The disease affection determination device 100 may include a control circuit which controls each function. Each circuit may be mounted by an ASIC (application specific integrated circuit), an FPGA (field programmable gate array), or the like.

In the entire description, at least a part of the device and the system may be constituted by hardware, or by software, and a CPU (Central Processing Unit) or the like may implement information processing of the software. When at least a part of the device and the system is constituted by the software, programs that implement the device, the system, and at least a part of the functions may be stored in a storage medium, such as a flexible disk or a CD-ROM, and may be executed by being read by a computer. The storage medium may be a detachable medium such as a magnetic disk or an optical disk, and also may be a fixed storage medium such as a hard disk device or a memory. In other words, the information processing by software may be concretely implemented using hardware resources. Further, the processing by software may be implemented on a circuit such as the FPGA, and may be executed by hardware. A job may be executed using, for example, an accelerator such as a GPU (Graphics Processing Unit).

For example, the computer can be used for the device of the above-described embodiments by reading dedicated software stored in a computer-readable storage medium. Any storage medium can be used. The computer may be used for the device of the above-described embodiments by installing the dedicated software downloaded via a communication network. In this way, the information processing by software is concretely implemented using hardware resources.

In the embodiments described above, an example has been described in which the program is executed by one processor, but the program may be executed by two or more processors. Therefore, the program may be in an aspect in which not only one program but also several programs are collectively used.

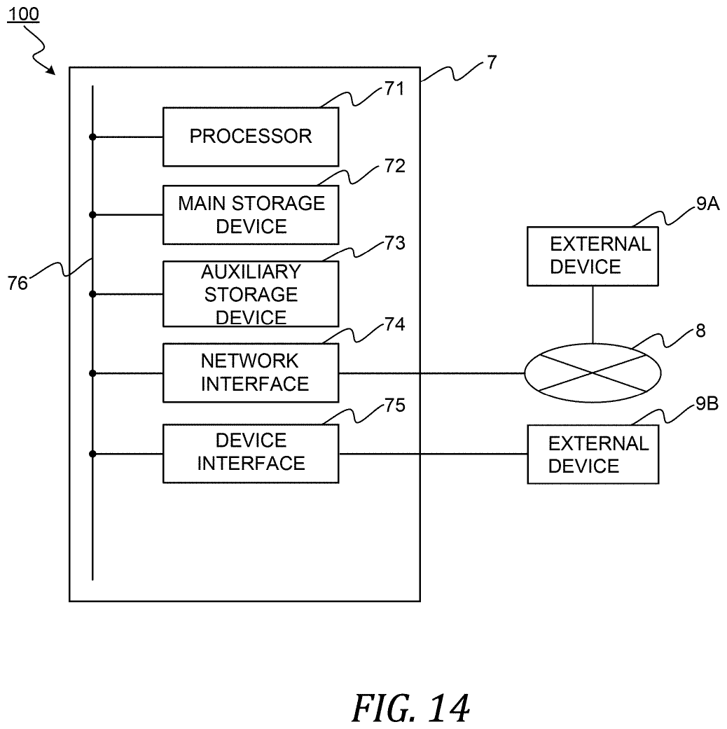

FIG. 14 is a block diagram showing an example of a hardware configuration according to the embodiments of the present disclosure. The device and the system according to the embodiments described above can be realized as a computer device 7 including a processor 71, a main storage device 72, an auxiliary storage device 73, a network interface 74, and a device interface 75 which are connected to each other via a bus 76.

The computer device 7 shown in FIG. 14 includes one for each of the components, but may include a plurality of the same components. Further, although one computer device 7 is shown, the software may be installed into a plurality of computer devices, and each of the plurality of computer devices may execute some different processes of the software.

The processor 71 may be an electronic circuit (processing circuit or processing circuitry) including a control device and an arithmetic device of the computer. The processor 71 may perform arithmetic processing based on data and programs input from each device of an internal configuration of the computer device 7, and output arithmetic operation results and control signals to each device. Specifically, the processor 71 may control each component constituting the computer device 7 by executing an OS (operating system) or an application of the computer device 7. The processor 71 may be any processor capable of performing the above processing. The device, the system, and each components of the device and the system are realized by the processor 71. Here, the processing circuit may be one or more electric circuits arranged on one chip, or may be one or more electric circuits arranged on two or more chips or devices.

The main storage device 72 is a storage device that stores instructions executed by the processor 71, various data, and the like, and information stored in the main storage device 72 is directly read by the processor 71. The auxiliary storage device 73 is a storage device other than the main storage device 72. These storage devices mean any electronic components capable of storing electronic information, and may be a memory or a storage. The memory includes both or one of a volatile memory and a nonvolatile memory. The memory storing various data in the device and the system, for example, the storage unit 30 may be realized by the main storage device 72 or the auxiliary storage device 73. For example, at least a part of the storage units described above may be mounted on the main storage device 72 or the auxiliary storage device 73. As another example, when an accelerator is provided, at least a part of the storage units described above may be mounted in a memory provided in the accelerator.

The network interface 74 is an interface used to connect to a communication network 8 in a wired or wireless manner. An interface compatible with an existing communication protocol may be used as the network interface 74. The network interface 74 may exchange information with an external device 9A which is in communication with computer device 7 via the communication network 8.

The external device 9A may include, for example, a camera, a motion capture device, an output destination device, an external sensor, and an input source device. In addition, the external device 9A may be a device having a function of some components of the disease affection determination device 100. The computer device 7 may receive a part of processing results of the disease affection determination device 100 via the communication network 8 such as a cloud service. Further, the server may be connected to the communication network 8 as the external device 9A, and the trained model may be stored in the server as the external device 9A. In this case, the disease affection determination device 100 may access the server as the external device 9A via the communication network 8 to perform the disease affection determination.

The device interface 75 may be an interface such as a USB (universal serial bus) which directly connects with an external device 9B. The external device 9B may be an external storage medium or a storage device. Each of the storage units may be realized by the external device 9B.

The external device 9B may be an output device. The output device may be, for example, a display device to display images, or may be a device for outputting voice. For example, there are an LCD (liquid crystal display), a CRT (cathode ray tube), a PDP (plasma display panel), and a speaker, but the present disclosure is not limited thereto.

The external device 9B may include an input device. The input device may include devices such as a keyboard, a mouse, and a touch panel, and may supply information input by these devices to the computer device 7. Signals from the input device are output to the processor 71.

### Overview of Embodiments

(1) The training device of the present disclosure includes a machine learning unit that trains a model for a predetermined disease using, as an input, a training feature vector based on an appearance frequency of a plurality of types of substrings in a base sequence obtained from a training sample collected from a learning subject and, as an output, label information indicating whether the learning subject is a subject affected by the predetermined disease or a subject not affected by the predetermined disease.

Since the model is trained using the training feature vector described above as the input and the label information described above as the output, it is possible to obtain a model for determining disease affection for a predetermined disease without time-consuming mapping. In addition, since mapping is not performed, it is possible to obtain a model for determining disease affection for a predetermined disease, for various living organisms other than humans.

(2) In the training device of (1), the base sequence may be acquired as a DNA sequence using a DNA sequencer by obtaining a corresponding DNA or RNA from the training sample. In this case, RNA sequence data, which is a base sequence, is obtained as the output of the DNA sequencer. Therefore, it is possible to obtain the appearance frequency of the plurality of types of substrings in the RNA sequence data, and it is possible to use the training feature vector based on the appearance frequency.

(3) In the training device of (1) or (2), the plurality of types of substrings may be extracted from a training read, which is a string having a predetermined length representing the base sequence. In this case, since the training read is the string having the predetermined length representing the base sequence, it is possible to obtain the appearance frequency of the plurality of types of substrings in the read, and it is possible to use the training feature vector based on the appearance frequency.

(4) In the training device of any one of (1) to (3), the appearance frequency of the plurality of types of substrings may be normalized. In this case, even when the amount of data of the training sample is different for each sample, the appearance frequency of the plurality of types of substrings is normalized, so that the difference in the appearance frequency due to the difference in the amount of data is corrected.

(5) In the training device of any one of (1) to (4), the substring may be k-mer. In this case, the substring composed of a continuous base cut out for each number of characters k is obtained in the base sequence represented as a string of a predetermined length. Since the substring repeatedly may appear in the base sequence, it is possible to obtain the appearance frequency of the substring, and it is possible to use the training feature vector based on the appearance frequency.

(6) In the training device of any one of (1) to (4), the substring may be a substring in which some of continuous characters included in the base sequence obtained from the training sample are skipped. In this case, since the substring is a part of continuous characters, that is, some characters are skipped, it is possible to make the disease affection determination robust against difference in RNA sequences due to individual differences in samples and sequencing errors.

(7) In the training device of (5) to (6), the substring may be a string in which a partially different string is converted into the same string using an error correcting code. In this case, the difference in RNA sequences due to the individual differences in the samples and the sequencing errors are further absorbed, and the disease affection determination is performed robustly.

(8) A disease affection determination device of the present disclosure includes a disease affection determination unit that uses, as an input, a feature vector for determination based on an appearance frequency of a plurality of types of substring in a base sequence obtained from a biological sample for determination collected from a determination subject for a predetermined disease to perform a disease affection determination for the predetermined disease on the determination subject.

Since the disease affection determination is performed on the determination subject using the feature vector for determination described above as the input, the disease affection determination for a predetermined disease is performed without time-consuming mapping. In addition, since mapping is not performed, the affection determination for a predetermined disease is performed on various living organisms other than humans.

(9) In the disease affection determination device of (8), the base sequence may be acquired as a DNA sequence using a DNA sequencer by obtaining a corresponding DNA from the determination sample. In this case, RNA sequence data, which is a base sequence, is obtained as the output of the DNA sequencer. Therefore, it is possible to obtain the appearance frequency of the plurality of types of substrings in the RNA sequence data, and it is possible to use the feature vector for determination based on the appearance frequency.

(10) In the disease affection determination device of (8), the appearance frequency of the plurality of types of substrings may be normalized. In this case, even when the amount of data of the determination sample is different for each sample, the appearance frequency of the plurality of types of substrings is normalized, so that the difference in the appearance frequency due to the difference in the amount of data is corrected.

(11) In the disease affection determination device of any of (8) to (10), the substring may be k-mer. In this case, the substring composed of a continuous base cut out for each number of characters k is obtained in the base sequence represented as a string of a predetermined length. Since the substring repeatedly may appear in the base sequence, it is possible to obtain the appearance frequency of the substring, and it is possible to use the feature vector for determination based on the appearance frequency.

(12) A machine learning method of the present disclosure includes: a step of inputting a training feature vector based on an appearance frequency of a plurality of types of substrings in a base sequence obtained from a training sample collected from a learning subject for a predetermined disease; and a step of training a model using, as an output, label information indicating whether the learning subject is a subject affected by the predetermined disease or a subject not affected by the predetermined disease.

Since the model is trained using the training feature vector described above as the input and the label information described above as the output, it is possible to train a model for determining disease affection for a predetermined disease without time-consuming mapping. In addition, since mapping is not performed, it is possible to train a model for determining disease affection for a predetermined disease, for various living organisms having no reference genome other than humans.

(13) The present disclosure is realized as a program for causing a computer to function as the training device. The training device is implemented by execution of the computer by the program of the present disclosure.

(14) The present disclosure is realized as a program for causing a computer to function as the disease affection determination device. The disease affection determination device is implemented by execution of the computer by the program of the present disclosure.

A person skilled in the art may come up with addition, effects or various kinds of modifications of the present disclosure based on the above-described entire description, but aspects of the present disclosure are not limited to the above-described individual embodiments. Various kinds of addition, changes and partial deletion can be made within a range that does not depart from the conceptual idea and the gist of the present disclosure derived from the contents specified in claims and equivalents thereof. For example, in all the above-described embodiments, the numerical values used in the description are merely an example, and are not limited thereto.

The present disclosure is not limited to the above-described embodiments, and various improvements and design changes can be made without departing from the gist of the present disclosure. These novel embodiments can be implemented in various other embodiments, and various omissions, replacements, and changes can be made without departing from the gist of the invention. These embodiments and modifications thereof are included in the scope and gist of the invention, and are also included in the scope of the invention described in claims and the equivalent scope thereof.

### <Supplementary Note>

In addition, for example, the embodiments of the present disclosure may be the following method or recording medium.

(1) A method of classifying a determination subject into a first clinical condition,

in a computer system including one or more processors and one or more memories storing one or more programs, the one or more programs solely or collectively including:

a) an instruction to obtain a plurality of sequence reads in electronic form from an unencoded ribonucleic acid molecule in the biological sample of the determination subject;

b) an instruction to extract one or more substrings from each sequence read in the plurality of sequence reads and obtain a plurality of substrings;

c) an instruction to determine an observed appearance frequency of each substring type in a series of substring types; and

d) an instruction to apply the observed appearance frequency of each substring type to a trained classification, wherein

the trained classification provides a possibility that the determination subject has the first clinical condition.

(2) The method according to Supplementary note (1), wherein the instruction of c) further includes an instruction to determine a substantial amount of the plurality of substrings located in each substring type in the series of substring types.

(3) The method according to Supplementary note (1), wherein the instruction of d) further includes an instruction to compare the observed appearance frequency of the individual substring types in the series of substring types with an appearance frequency of reference substrings corresponding to the individual substring types.

(4) The method according to Supplementary note (1), wherein the plurality of sequence reads are obtained from single-ended next-generation sequencing or pair-ended next-generation sequencing for the biological sample of the determination subject.

(5) The method according to Supplementary note (1), wherein each sequence read in the plurality of sequence reads is a sequence read of all or partial mircroRNA from the biological sample.

(6) The method according to Supplementary note (1), wherein the observed appearance frequency of the individual substring types in the series of substring types is normalized.

(7) The method according to any one of Supplementary notes (1) to (6), wherein each substring in the series of substring types is a k-mer having a first predetermined length of a nucleic acid residue.

(8) The method according to any one of Supplementary notes (1) to (6), wherein the plurality of types of substrings include one or more substrings having a first predetermined length and one or more substrings having a second predetermined length for each sequence read in the plurality of sequence reads.

(9) The method according to Supplementary note (7) or (8), wherein each of the first predetermined length and the second predetermined length is individually selected from at least one residue, at least two residues, at least three residues, at least four residues, at least five residues, at least six residues, at least seven residues, at least eight residues, at least nine residues, at least ten residues, at least eleven residues, at least twelve residues, or at least fifteen residues.

(10) The method according to any one of Supplementary notes (1) to (6), wherein each substring type in the series of substring types includes a discontinuous string of nucleic acid residues from the individual sequence reads in the plurality of sequence reads.

(11) The method according to any one of Supplementary notes (1) to (6), wherein each substring type in the series of substring types include a different type of string that is converted into a similar type of string using an error correcting code.

(12) The method according to any one of Supplementary notes (1) to (11), wherein the determination subject is a human.

(13) The method according to any one of Supplementary notes (1) to (12), wherein the first clinical condition is cancer from a common primary site.

(14) The method according to any one of Supplementary notes (1) to (12), wherein the first clinical condition is cancer from two or more common primary sites.

(15) The method according to any one of Supplementary notes (1) to (12), wherein the first clinical condition is breast cancer, lung cancer, prostate cancer, colorectal cancer, renal cancer, uterine cancer, pancreatic cancer, esophageal cancer, lymphoma, head/neck cancer, ovarian cancer, hepatobiliary cancer, melanoma, cervical cancer, multiple myeloma, leukemia, thyroid cancer, bladder cancer, stomach cancer, or a combination thereof.

(16) The method according to any one of Supplementary notes (1) to (13), wherein the first clinical condition is a predetermined stage of breast cancer, a predetermined stage of lung cancer, a predetermined stage of prostate cancer, a predetermined stage of colorectal cancer, a predetermined stage of renal cancer, a predetermined stage of uterine cancer, a predetermined stage of pancreatic cancer, a predetermined stage of esophageal cancer, a predetermined stage of lymphoma, a predetermined stage of head/neck cancer, a predetermined stage of ovarian cancer, a predetermined stage of hepatobiliary cancer, a predetermined stage of melanoma, a predetermined stage of cervical cancer, a predetermined stage of multiple myeloma, a predetermined stage of leukemia, a predetermined stage of thyroid cancer, a predetermined stage of bladder cancer, or a predetermined stage of stomach cancer.

(17) The method according to any one of Supplementary notes (1) to (13), wherein the first clinical condition is a predetermined subtype of cancer.

(18) The method according to Supplementary note (17), wherein the cancer is breast cancer, lung cancer, prostate cancer, colorectal cancer, renal cancer, uterine cancer, pancreatic cancer, esophageal cancer, lymphoma, head/neck cancer, ovarian cancer, hepatobiliary cancer, melanoma, cervical cancer, multiple myeloma, leukemia, thyroid cancer, bladder cancer, or stomach cancer.

(19) The method according to any one of Supplementary note (1) to (18), wherein the biological sample is blood, whole blood, lymph, serum, saliva, urine, cerebrospinal fluid, fine needle aspiration fluid, tissue specimen, breast milk, nipple discharge, or in vitro fluid of the determination subject.

(20) A classification device including one or more processors and one or more memories storing one or more programs to be executed by the one or more processors, the one or more programs solely or collectively including:

a) an instruction to obtain a plurality of sequence reads in electronic form from an unencoded ribonucleic acid molecule in the biological sample of the determination subject;

b) an instruction to extract one or more substrings from each sequence read in the plurality of sequence reads and obtain a plurality of substrings;

c) an instruction to determine an observed appearance frequency of each substring type in a series of substring types; and

d) an instruction to apply the observed appearance frequency of each substring type to a trained classification, wherein

the trained classification provides a possibility that the determination subject has the first clinical condition.

(21) A non-transitory computer-readable recording medium in which one or more computer programs are embedded for classification, the one or more programs causing a computer system to execute a method for the classification when being executed by the computer system and solely or collectively including:

a) an instruction to obtain a plurality of sequence reads in electronic form from an unencoded ribonucleic acid molecule in the biological sample of the determination subject;

b) an instruction to extract one or more substrings from each sequence read in the plurality of sequence reads and obtain a plurality of substrings;

c) an instruction to determine an observed appearance frequency of each substring type in a series of substring types; and

d) an instruction to apply the observed appearance frequency of each substring type to a trained classification, wherein

the trained classification provides a possibility that the determination subject has the first clinical condition.

(22) A classification method, in a computer system including one or more processors and one or more memories storing one or more programs to be executed by the one or more processors,

the classification method including:

a) for an individual reference subject in a plurality of reference subjects, the individual reference subject in the plurality of reference subjects including a corresponding clinical condition label from a plurality of clinical condition labels,

obtaining a plurality of sequence reads in electronic form from an unencoded ribonucleic acid molecule in a biological sample of the individual reference subject;

extracting one or more substrings from each sequence read in the plurality of sequence reads and obtaining a plurality of corresponding reference substrings; and

determining a reference appearance frequency of each substring type in a series of substring types, using the plurality of corresponding reference substrings; and

b) training an untrained or partially trained classification for the individual reference appearance frequency of each substring type and the corresponding clinical condition label of the individual reference subject in the plurality of reference subjects, and obtaining a trained classification that identifies the plurality of clinical condition labels based on a large number of unencode ribonucleic acid molecules.

(23) The classification method according to Supplementary note (22), wherein the individual reference subject in the plurality of reference subjects is a human.

(24) The classification method according to Supplementary note (22) or (23), wherein the plurality of reference subjects includes at least 20 subjects.

(25) The classification method according to Supplementary note (22) or (23), wherein the plurality of reference subjects includes at least 100 subjects.

(26) The classification method according to any one of Supplementary notes (22) to (25), wherein the obtaining the plurality of sequence reads in electronic form further includes obtaining the biological sample of the reference subject and generating the plurality of corresponding sequence reads.

(27) The classification method according to any one of Supplementary notes (22) to (26), wherein the plurality of clinical condition labels include two or more clinical conditions selected from the group including breast cancer, lung cancer, prostate cancer, colorectal cancer, renal cancer, uterine cancer, pancreatic cancer, esophageal cancer, lymphoma, head/neck cancer, ovarian cancer, hepatobiliary cancer, melanoma, cervical cancer, multiple myeloma, leukemia, thyroid cancer, bladder cancer, or stomach cancer.

(28) The classification method according to any one of Supplementary notes (22) to (26), wherein the plurality of clinical conditions includes two or more clinical conditions selected from the group including a predetermined stage of breast cancer, a predetermined stage of lung cancer, a predetermined stage of prostate cancer, a predetermined stage of colorectal cancer, a predetermined stage of renal cancer, a predetermined stage of uterine cancer, a predetermined stage of pancreatic cancer, a predetermined stage of esophageal cancer, a predetermined stage of lymphoma, a predetermined stage of head/neck cancer, a predetermined stage of ovarian cancer, a predetermined stage of hepatobiliary cancer, a predetermined stage of melanoma, a predetermined stage of cervical cancer, a predetermined stage of multiple myeloma, a predetermined stage of leukemia, a predetermined stage of thyroid cancer, a predetermined stage of bladder cancer, or a predetermined stage of stomach cancer.

(29) The classification method according to Supplementary note (27) or (28), wherein the plurality of clinical condition labels further includes a healthy state.

(30) The classification method according to any one of Supplementary notes (22) to (29), wherein the trained classification is a neural network algorithm, a support vector machine algorithm, a decision tree algorithm, an unsupervised clustering model algorithm, a supervised clustering model algorithm, or a regression model.

(31) The classification method according to any one of Supplementary notes (22) to (30), wherein the number of the trained classifications is 2 or more.

(32) The classification method according to any one of Supplementary notes (22) to (30), wherein the number of the trained classifications is 2.

(33) A classification device including one or more processors and one or more memories storing one or more programs to be executed by the one or more processors,

the one or more programs solely or collectively including:

a) for an individual reference subject in a plurality of reference subjects, the individual reference subject in the plurality of reference subjects including a corresponding clinical condition label from a plurality of clinical condition labels,

an instruction to obtain a plurality of sequence reads in electronic form from an unencoded ribonucleic acid molecule in a biological sample of the individual reference subject;

an instruction to extract one or more substrings for each sequence read in the plurality of sequence reads and obtain a plurality of corresponding reference substrings; and

an instruction to determine a reference appearance frequency of each substring type in a series of substring types, using the plurality of corresponding reference substrings; and

b) an instruction to train an untrained or partially trained classification for the individual reference appearance frequency of each substring type and the corresponding clinical condition label of the individual reference subject in the plurality of reference subjects, and obtain a trained classification that identifies the plurality of clinical condition labels based on a large number of unencode ribonucleic acid molecules.

(34) A non-transitory computer-readable recording medium in which one or more computer programs are embedded for classification, the one or more programs causing a computer system to execute a method for classification when being executed by the computer system,

the method for classification including:

a) for an individual reference subject in a plurality of reference subjects, the individual reference subject in the plurality of reference subjects including a corresponding clinical condition label from a plurality of clinical condition labels,

obtaining a plurality of sequence reads in electronic form from an unencoded ribonucleic acid molecule in a biological sample of the individual reference subject;

extracting one or more substrings from each sequence read in the plurality of sequence reads and obtaining a plurality of corresponding reference substrings; and

determining a reference appearance frequency of each substring type in a series of substring types, using the plurality of corresponding reference substrings; and

b) training an untrained or partially trained classification for the individual reference appearance frequency of each substring type and the corresponding clinical condition label of the individual reference subject in the plurality of reference subjects, and obtaining a trained classification that identifies the plurality of clinical condition labels based on a large number of unencode ribonucleic acid molecules.

### REFERENCE SIGNS LIST

10 training device
11 machine learning unit
20 disease affection determination unit
30 storage unit
100 disease affection determination device
101 CPU
102 RAM
103 ROM
104 input device
105 communication interface
106 auxiliary storage device
107 output device
200 training phase
201 RNA sequence data
202 header line
203 sequence string
204 label information
205 sample ID
206 label
207 read
208 k-mer
209 appearance frequency
210 evaluation method
211 score
212 substring
213 substring
214 representative string
215 label
216 label
300 determination phase

1. A training device comprising at least one memory; and at least one processor configured to: train a model for a predetermined disease using, as an input, a training data based on an appearance frequency of a plurality of types of substrings in a base sequence obtained from a training sample collected from a learning subject and, as an output, label information indicating whether or not the learning subject is a subject affected by the predetermined disease.

2. A method of classifying a determination subject into a first clinical condition,
in a computer system including one or more processors and one or more memories storing one or more programs, the one or more programs solely or collectively comprising:
a) an instruction to obtain a plurality of sequence reads in electronic form from an unencoded ribonucleic acid molecule in the biological sample of the determination subject;
b) an instruction to extract one or more substrings from each sequence read in the plurality of sequence reads and obtain a plurality of substrings;
c) an instruction to determine an observed appearance frequency of each substring type in a series of substring types; and
d) an instruction to apply the observed appearance frequency of each substring type to a trained classification, wherein
the trained classification provides a possibility that the determination subject has the first clinical condition.

3. The method according to claim 2, wherein the instruction of c) further comprises an instruction to determine a substantial amount of the plurality of substrings located in each substring type in the series of substring types.

4. The method according to claim 2, wherein the instruction of d) further comprises an instruction to compare the observed appearance frequency of the individual substring types in the series of substring types with an appearance frequency of reference substrings corresponding to the individual substring types.

5. The method according to claim 2, wherein the plurality of sequence reads are obtained from single-ended next-generation sequencing or pair-ended next-generation sequencing for the biological sample of the determination subject.

6. The method according to claim 2, wherein each sequence read in the plurality of sequence reads is a sequence read of all or partial mircroRNA from the biological sample.

7. The method according to claim 2, wherein the observed appearance frequency of the individual substring types in the series of substring types is normalized.

8. The method according to claim 2, wherein each substring in the series of substring types is a k-mer having a first predetermined length of a nucleic acid residue.

9. The method according to claim 2, wherein the plurality of types of substrings comprises one or more substrings having a first predetermined length and one or more substrings having a second predetermined length for each sequence read in the plurality of sequence reads.

10. The method according to claim 8, wherein each of the first predetermined length and the second predetermined length is individually selected from at least one residue, at least two residues, at least three residues, at least four residues, at least five residues, at least six residues, at least seven residues, at least eight residues, at least nine residues, at least ten residues, at least eleven residues, at least twelve residues, or at least fifteen residues.

11. The method according to claim 2, wherein each substring type in the series of substring types comprises a discontinuous string of nucleic acid residues from the individual sequence reads in the plurality of sequence reads.

12. The method according to claim 2, wherein each substring type in the series of substring types includes a different type of string that is converted into a similar type of string using an error correcting code.

13. A classification method, in a computer system including one or more processors and one or more memories storing one or more programs to be executed by the one or more processors,
the classification method comprising:
a) for an individual reference subject in a plurality of reference subjects, the individual reference subject in the plurality of reference subjects including a corresponding clinical condition label from a plurality of clinical condition labels,
obtaining a plurality of sequence reads in electronic form from an unencoded ribonucleic acid molecule in a biological sample of the individual reference subject;
extracting one or more substrings from each sequence read in the plurality of sequence reads and obtaining a plurality of corresponding reference substrings; and
determining a reference appearance frequency of each substring type in a series of substring types, using the plurality of corresponding reference substrings; and
b) training an untrained or partially trained classification for the individual reference appearance frequency of each substring type and the corresponding clinical condition label of the individual reference subject in the plurality of reference subjects, and obtaining a trained classification that identifies the plurality of clinical condition labels based on a large number of unencode ribonucleic acid molecules.

14. The classification method according to claim 13, wherein the trained classification is a neural network algorithm, a support vector machine algorithm, a decision tree algorithm, an unsupervised clustering model algorithm, a supervised clustering model algorithm, or a regression model.

15. A classification device comprising one or more processors and one or more memories storing one or more programs to be executed by the one or more processors,
the one or more programs solely or collectively including:
a) for an individual reference subject in a plurality of reference subjects, the individual reference subject in the plurality of reference subjects including a corresponding clinical condition label from a plurality of clinical condition labels,
an instruction to obtain a plurality of sequence reads in electronic form from an unencoded ribonucleic acid molecule in a biological sample of the individual reference subject;
an instruction to extract one or more substrings from each sequence read in the plurality of sequence reads and obtain a plurality of corresponding reference substrings; and
an instruction to determine a reference appearance frequency of each substring type in a series of substring types, using the plurality of corresponding reference substrings; and
b) an instruction to train an untrained or partially trained classification for the individual reference appearance frequency of each substring type and the corresponding clinical condition label of the individual reference subject in the plurality of reference subjects, and obtain a trained classification that identifies the plurality of clinical condition labels based on a large number of unencode ribonucleic acid molecules.

16. A disease affection determination device comprising at least one processor configured to: use, as an input, a training data based on an appearance frequency of a plurality of types of substring in a base sequence obtained from a biological sample for determination collected from a determination subject to perform a disease affection determination for predetermined disease on the determination subject.

17. The disease affection determination device according to claim 16, wherein the base sequence is acquired as a DNA sequence using a DNA sequencer by obtaining a corresponding DNA from the determination sample.

18. The disease affection determination device according to claim 16, wherein the appearance frequency of the plurality of types of substrings is normalized.

19. The disease affection determination device according to claim 16, wherein the substring is k-mer.

20. A machine learning method comprising:
a step of inputting a training feature vector based on an appearance frequency of a plurality of types of substrings in a base sequence obtained from a training sample collected from a learning subject for a predetermined disease; and
a step of training a model using, as an output, label information indicating whether the learning subject is a subject affected by the predetermined disease or a subject not affected by the predetermined disease.
